(19) United States
(12) Patent Application Publication
(10) Pub. No.: US 2004/0176760 A1
(43) Pub. Date: Sep. 9, 2004

(54) Electrosurgical apparatus with cooling device

(76) Inventor: Xue Hua Qiu, Xian City (CN)

Correspondence Address: GOODWIN PROCTER L.L.P, 103 EISENHOWER PARKWAY, ROSELAND, NJ, 07068, US

(21) Appl. No.: 10/717,692

(22) Filed: Nov. 20, 2003

(30) Foreign Application Priority Data
Mar. 5, 2003 (TW) 092104645

(51) Int. Cl. A61B 18/14

(52) U.S. Cl. 606/45; 606/49

(57) ABSTRACT

The present invention relates to an electrosurgical apparatus with a cooling device for removing undesired tissues, such as tumors. The electrosurgical apparatus can be used to remove tissue and to coagulate tissue at the same time.

### FIELD OF THE INVENTION

The present invention relates to an electrosurgical apparatus for removing tissue and coagulating tissue at the same time with a radio-frequency energy. The electrosurgical apparatus of the present invention is useful for treating tumors.

### BACKGROUND OF THE INVENTION

Dysfunctional uterine bleeding, uterine myomatosis and uterine adenomyomatosis are common gynecological diseases. The prevalence of dysfunctional uterine bleeding, uterine myomatosis and uterine adenomyomatosis among women of child-bearing age is about 22%, 20% and 1-5% respectively. The prevalence of uterine adenomyomatosis among infertile women is about 48%. These diseases may have symptoms ranging from pressure related symptoms from excessive enlargement of tumors, excessive long-term bleeding and severe pain, which may become psychological and physical burdens on the patient and affect the sexual life of the patient. The diseases may further lead to anemia or even induce cancers and become fatal in severe cases.

U.S. Pat. No. 5,902,251 which issued to Neil C. vanHooydonk on May 11, 1999 and is entitled “Transcervical intrauterine applicator for intrauterine hyperthermia” teaches a transcervical intrauterine catheter applicator for the hyperthermia/thermal therapy treatment of the uterus. This treatment employs an electromagnetic radiation (EMR) device for the medical treatment by above-normal temperatures of dysfunctional (abnormal) uterine bleeding. The vanHooydonk device is complicated and expensive.

U.S. Pat. No. 6,575,969 which issued to William J. Rittman, III, et al. on Jun. 10, 2003 and is entitled “Cool-tip radiofrequency thermosurgery electrode system for tumor ablation” teaches another hyperthermia surgical device which is also complicated and expensive.

### SUMMARY OF THE INVENTION

The object of the present invention is to provide an effective electrosurgical apparatus, which is structurally simple, easy to operate and causes little damage to the normal tissue around the tissues to be removed.

Another object of the present invention is to provide an electrosurgical apparatus which allows safe, quick and effective operation and minimizes pain in the patient.

A further object of the present invention is to provide an electrosurgical apparatus for treating uterine myomatosis, which can be inserted into the uterus through the vagina or the cervical canal to remove the uterine tumors.

In accordance with these and other objects of the invention there is provided an electrosurgical apparatus for tissue removal, comprising a substantially hollow elongated body terminating in a sharp closed cutting head; an insulative layer covering the elongated body; the elongated body forming a portion of a coolant path; a handle located at an end of the elongated body opposite from said cutting head; coolant inlet/outlet connectors connected to the handle for introducing a coolant into the coolant path of the elongated body and releasing the coolant therefrom; and a radio-frequency energy input connector connected to the elongated body. In one preferred embodiment the elongated body comprises two metal tubes arranged side-by-side. In another preferred embodiment the elongated body comprises two metal tubes with different diameters, one with a smaller diameter being disposed coaxially within the other with a bigger diameter.

In a further preferred embodiment there is provided an electrosurgical apparatus for tissue removal, comprising: a substantially hollow elongated body terminating in a closed cutting head; wherein the elongated body comprises a metal tube which is folded over to form a loop section and a fork section having two legs; an insulative layer covering the elongated body; the elongated body forming a portion of a coolant path; a handle located at an end of the elongated body opposite from said cutting head; coolant inlet/outlet connectors connected to the handle for introducing a coolant into the coolant path of the elongated body and releasing the coolant therefrom; and a radio-frequency energy input connector connected to the elongated body. In another preferred embodiment the two legs of the fork section are adhered to each other. The cutting head may comprise the loop section of the folded tube and may be arranged at an angle to the elongated body. The cutting head may comprise two flat metal tubes arranged side-by-side and sealed at the ends, the metal tubes being in fluid communication with and connected to the two metal tubes of the elongated body.

### BRIEF DESCRIPTION OF THE DRAWINGS

The invention will become more fully understood from the following detailed description taken in conjunction with the accompanying drawings, in which:

FIG. 1a is an illustrative drawing of Embodiment a;

FIG. 1b is an illustrative drawing of Embodiment b;

FIG. 2a is a cross-sectional view of the handle of Embodiment a;

FIG. 2b is a cross-sectional view of the handle of Embodiment b;

FIG. 3a is a cross-sectional view of the cutting head of Embodiment a;

FIG. 3b is a cross-sectional view of the cutting head of Embodiment b;

FIG. 4a is a side view of the cutting head of Embodiment a;

FIG. 4b is a side view of the cutting head of Embodiment b;

FIG. 5 illustrates the operation of Embodiment a;

FIG. 6a is a side view of Embodiment c;

FIG. 6b is a top view of Embodiment c;

FIG. 7 is a cross-sectional view of the elongated body of Embodiment c;

FIG. 8 illustrates the operation of Embodiment c.

### DETAILED DESCRIPTION OF THE INVENTION

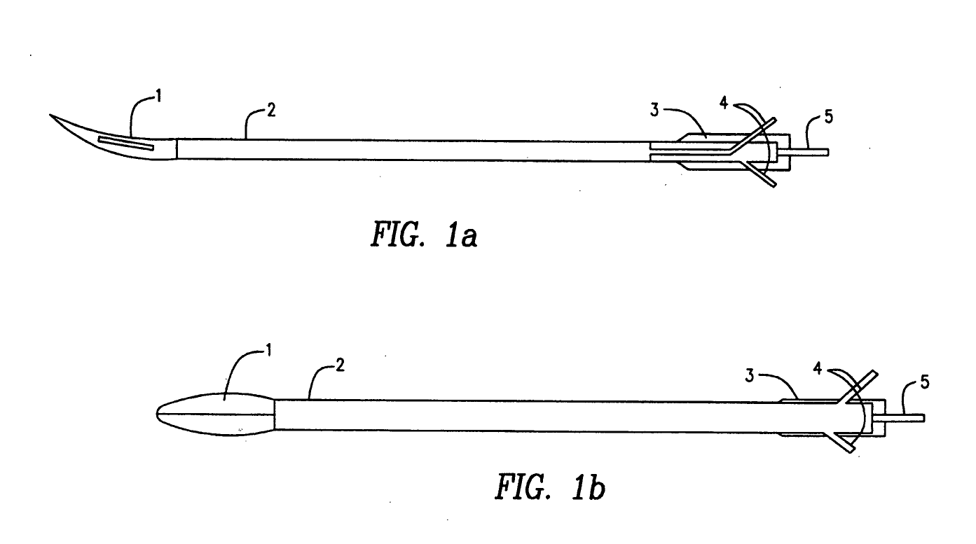

The present invention provides an electrosurgical apparatus with a cooling device for removing undesired tissue, such as tumors. As shown in FIG. 1, the electrosurgical apparatus of the present invention comprises an elongated body 2 terminating in a cutting head 1, a handle 3 having coolant inlet/outlet connectors 4 and a radio-frequency energy input connector 5, wherein the elongated body 2 has an insulative layer at the surface. The insulative layer may be a medical insulative paint or insulative tube surrounding the elongated body. The patient is placed on a conductive surface that can act as a ground electrode.

With the electrosurgical apparatus of the present invention, it is not necessary to remove the electrosurgical apparatus from the uterus several times during an operation to clean the cutting head from the tissue stuck on it. Therefore, multiple insertions of the electrosurgical apparatus can be avoided, and thus, increasing the convenience of operation, reducing the duration of operation and minimizing the damage to the normal tissues and the pain experienced by the patient. The duration of operation is short and the therapeutic efficacy is high so that hysterectomy can be avoided.

The electrosurgical apparatus of the present invention can be customized mostly at the cutting head and the elongated body depending upon the operation to be performed. Thus, a doctor may have a set of such devices. A proper electrosurgical apparatus is selected according to the characteristics of the tumors to be treated for easy positioning and penetration of the tissue, clear guidance by an ultrasound, and control of the form and amount of the radiofrequency energy emitted. The parts of the electrosurgical apparatus and the insulative layer must be stable under high temperatures (over 100° C.) and must not react with disinfectants, deform or release toxic substances. The insulative layer of the electrosurgical apparatus must be reliable with regard to its insulative characteristics under high temperatures or exposure to disinfectant solutions, and must be tolerant to physical impact and frictions. The insulative layer must not detach from the electrosurgical apparatus under high temperatures or exposure to disinfectant solutions.

The radiofrequency therapy system and the electrosurgical apparatus are suitable for removing undesired tissue in human cavities, such as benign or malignant tumors. They are particularly useful for the treatment of fibroid tumors, dysfunctional uterine bleeding, cervical erosion and cervical polyps. The fibroid tumors that can be treated by the radiofrequency therapy system and the electrosurgical apparatus of the present invention include cervical fibroids and submucous fibroids of various sizes, intramyometrial fibroids of a diameter of less than 5 cm, fibroids present in the cervical wall of a diameter of less than 5 cm, intracavitary fibroids of various sizes, and subserous fibroids of a diameter of less than 5 cm.

The following sections describe exemplary embodiments of the present invention. It should be apparent to those skilled in the art that the described embodiments of the present invention provided herein are illustrative only and not limiting, having been presented by way of example only. Therefore, numerous other embodiments of the modifications thereof are contemplated as falling within the scope of the present invention as defined herein and equivalents thereto.

### EXAMPLE 1

### Electrosurgical Apparatus With Cooling Device: Embodiments a and b

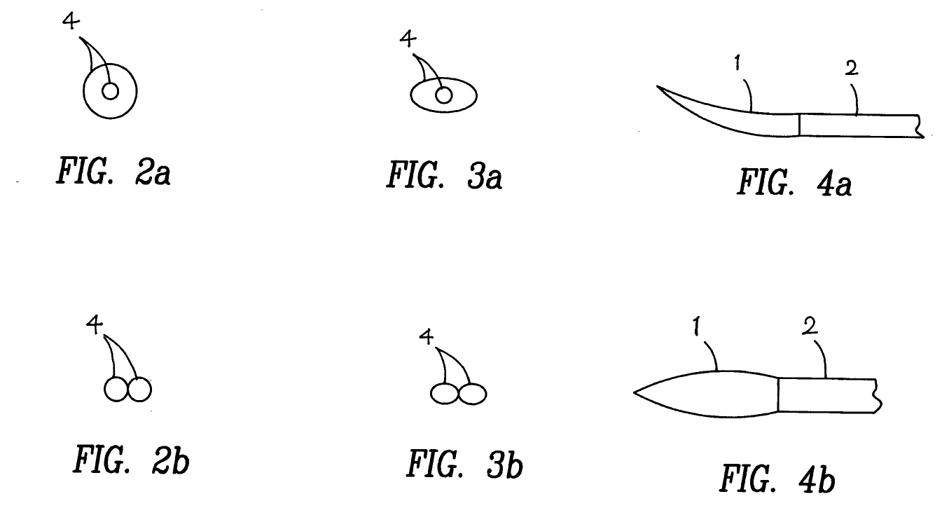

The electrosurgical apparatus with a cooling device for removing undesired tissue comprises, see FIGS. 1a and 1b, a substantially hollow elongated body 2, a cutting head 1, a radio-frequency energy input connector 5, a handle 3, and coolant inlet/outlet connectors 4 which extend from the handle 3. The elongated body 2 and the cutting head 1 comprise two metal tubes, see FIGS. 2a and 2b, while the elongated body has an insulative layer at its surface. The two metal tubes can be arranged side-by-side, see FIG. 2b, and welded together. Alternatively, the two metal tubes can have different diameters and the one with a smaller diameter is disposed coaxially within the other having a bigger diameter, see FIG. 2a. The elongated body is connected to and sealed with the coolant inlet/outlet connectors. The cutting head is a flat, substantially hollow metal tube. Alternatively, the cutting head comprises two tubes welded side-by-side and is of a curved or straight shape. The two tubes are welded at the distal ends to form a sharp, closed tip. The insulative layer of the elongated body 2 is a medical insulative paint or an insulative tube surrounding the elongated body.

In a preferred embodiment a, the elongated body 2 comprises two metal tubes having different diameters, see FIGS. 1a, 2a and 3a. The tube with a smaller diameter is disposed coaxially within the other tube having a bigger diameter. The smaller tube located within the bigger tube extrudes from the handle 3 and is welded and sealed with the bigger tube at the connecting point. The smaller tube serves as a coolant inlet/outlet connector 4 and is used for supplying the coolant. The bigger tube is welded with another smaller tube as a coolant inlet/outlet connector 4 for releasing the coolant, which is in fluid communication with the bigger tube and extrudes from the handle. An end of the bigger tube is sealed and connected to the radio-frequency energy input connector 5.

In a preferred embodiment b, the elongated body 2 comprises two metal tubes with substantially the same diameter arranged side-by-side, see FIGS. 1b, 2b and 3b. Each of the two metal tubes is welded with and connected to a coolant inlet/outlet connector 4, which is in fluid communication with the elongated body 2 and extrudes from the handle 3. The connecting points of each of the coolant inlet/outlet connectors and the elongated body are welded and sealed. The ends of the two metal tubes are sealed and are connected to the radio-frequency energy input connector 5 for transmitting the radio-frequency energy to the cutting head.

In the preferred embodiment a, the cutting head is not covered by an insulative layer, see FIGS. 4a and 4b. The ends of the tubes at the tip of the cutting head are welded and sealed. The cutting head may be curved, straight or of any desired shape. The distal end of the cutting head is a sharp tip. The inner smaller tube extends from the connecting point with the coolant inlet/outlet connector 4 all the way to the tip of the cutting head. In the preferred embodiment b, the cutting head 1 is formed from connecting, welding and sealing the ends of the two tubes arranged side-by-side and may be curved, straight of any desired shape. The distal end of the cutting head is sharp. The two tubes within the tip of cutting head are in fluid communication with each other.

The handle 3 is fixed to the elongated body 2 and is provided with coolant inlet/outlet connectors 4, which are connected to a coolant generator through a medical catheter for transporting the coolant to the cutting head 1 through the elongated body 2 to achieve the cooling effect. The rear end of the handle 3 has a radio-frequency energy input connector 5, which is to be connected with a radio-frequency energy generator.

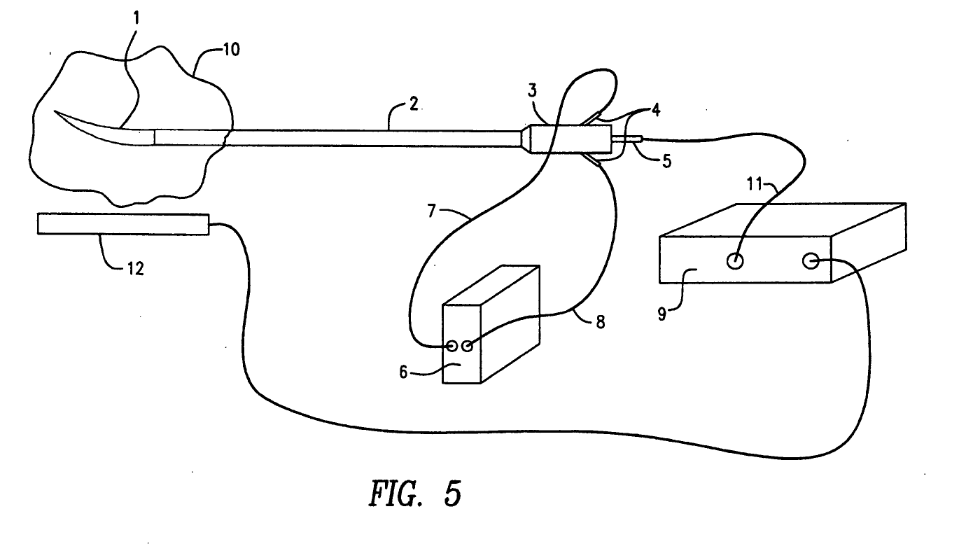

As shown in FIG. 5, during operation, a coolant generator 6, coolant delivery tubes 7, 8 are respectively connected to the coolant inlet/outlet connectors 4 on the handle 3. The radio-frequency energy input connector 5 and the radio-frequency energy generator 9 are connected. With the guidance of an ultrasound, the cutting head 1 is inserted through the vagina or the cervical canal into the uterine tumor 10 to be removed. Then the radio-frequency energy generator 9 and the coolant generator 6 are switched on so that the radio-frequency energy 11 flows from the radio-frequency energy generator through the connector 5, the handle 3 and the elongated body 2 to the cutting head 1 and acts on the tumor tissue before returning to grounding sheet 12. High degrees of heat generated instantaneously solidifies, denatures and damages the tumor tissue to achieve the therapeutic effect. At the same time, through the pumping action of the coolant generator, the coolant is continuously supplied from a coolant inlet/outlet connector 4 to the cutting head 1 through the elongated body 2 and exits from the other coolant inlet/outlet connector 4. Therefore, the coolant can circulate in the electrosurgical apparatus to cool the cutting head, and thereby, the tissue does not stick to the cutting head during operation. Thus, the need for removing the cutting head from the uterus to clean off the tissues stuck on it several times during operation can be eliminated and therefore the operation procedure is simplified. Furthermore, by using the cooling system 6, the tissues stay on the surface of the cutting head longer without being completely dehydrated and thus, the energy output time is extended. Therefore, a bigger tumor can be treated with one insertion of the electrosurgical apparatus. The inconvenience associated with multiple insertions of the apparatus can be avoided.

### EXAMPLE 2

### Electrosurgical Apparatus With Cooling Device: Embodiment c

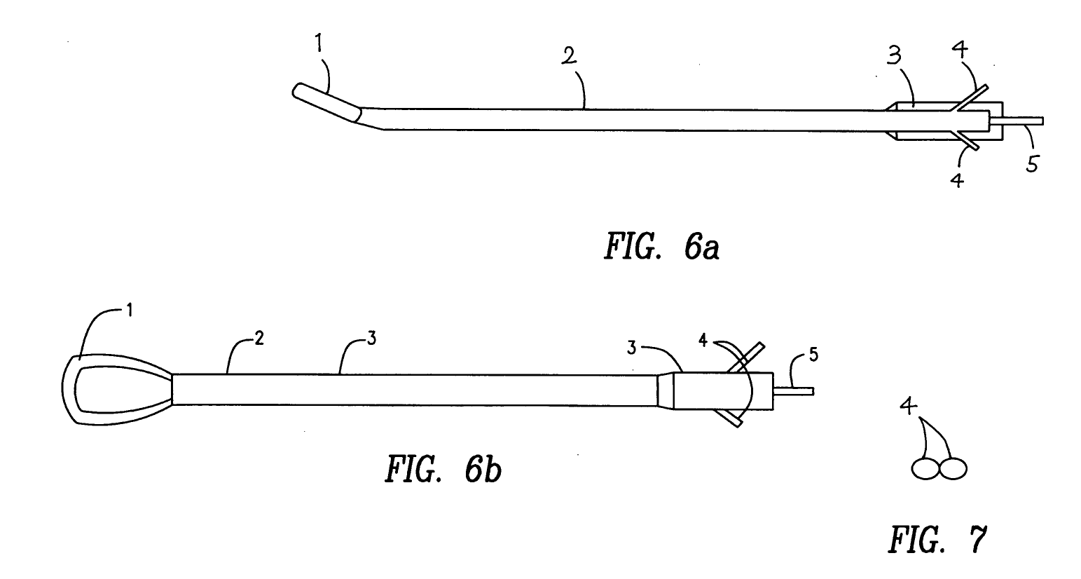

In an exemplary embodiment c, see FIGS. 6a, 6b and 7, the electrosurgical apparatus with a cooling device for removing undesired tissues comprises a substantially hollow elongated body, a cutting head at the dismal end of the elongated body, and a handle which is located at the proximal end of the elongated body and has a radio-frequency energy input connector and coolant inlet/outlet connectors. The elongated body is made by folding a metal tube over to form a loop and a fork with two legs arranged-side-by-side and welding the two legs together. The elongated body has an insulative layer at the surface and is connected to and sealed with the coolant inlet/outlet connectors. The cutting head comprises the loop section of the folded metal tube and is arranged at an angle to the elongated body. The insulative layer of the elongated body is a medical insulative paint or an insulative tube surrounding the elongated body.

As shown in FIGS. 6a and 6b, the electrosurgical apparatus of the present invention comprises an elongated body 2 with a cutting head 1 and a handle 3 having coolant inlet/outlet connectors 4 and a radio-frequency energy input connector 5. The elongated body 2 is made by folding over a metal tube to form a loop and a fork with two legs arranged side-by-side and welding the two legs together. The elongated body has an insulative layer at its surface. The insulative layer may be a medical insulative paint or an insulative tube surrounding the elongated body. Each of the two legs is welded with one of the coolant input/output connectors 4. Both of the coolant input/output connectors extends from the handle 3 and are connected to the radio-frequency energy input connector 5.

The cutting head 1 comprises the loop section of the folded metal tube forming the elongated body 2, but does not have an insulative layer. The cutting head is arranged at an angle to the elongated body 2. The whole metal tube is in fluid communication internally.

The handle 3 is fixed to the elongated body 2 and has coolant inlet/outlet connectors 4, each of which is connected to the coolant generator through a medical catheter for transporting the coolant to the cutting head 1 through the elongated body 2 to achieve the cooling effect. The end of the handle 3 has a radio-frequency energy input connector 5, which is to be connected to a radio-frequency energy generator for transmitting a radio-frequency energy from the radio-frequency energy input connector 5 through the elongated body 2 to the cutting head 1.

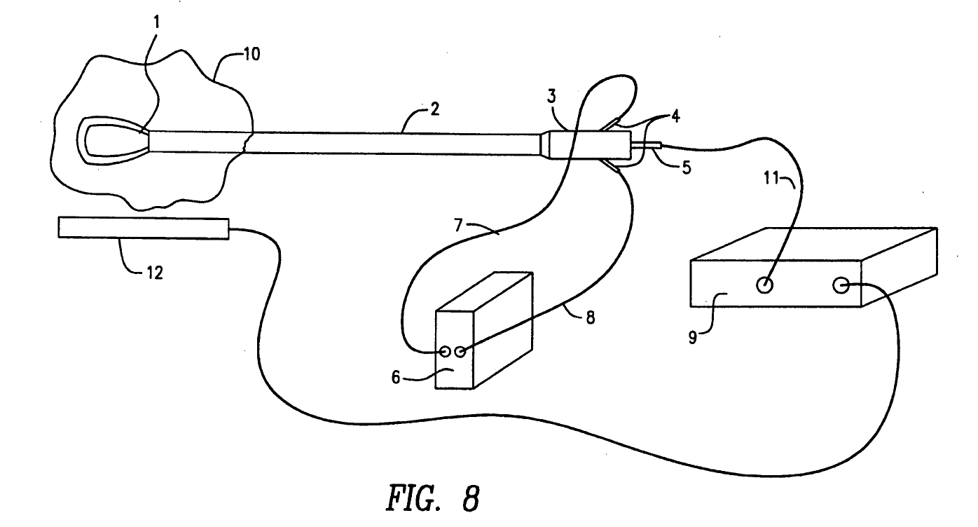

As shown in FIG. 8, during operation, the coolant generator 6, coolant delivery tubes 7, 8 are respectively connected to the coolant inlet/outlet connectors of the handle 3. The radio-frequency energy input connector 5 and the radio-frequency energy generator 11 are connected. The cutting head is inserted through the vagina or the cervical canal to a starting point for removing undesired tissues 10. Then the radio-frequency energy generator and the coolant generator are switched on so that the radio-frequency energy 11 flows from the radio-frequency energy generator 9 through the radio-frequency energy input connector 5, the handle 3, the elongated body 2 to the cutting head 1 and acts on the undesired tissue. At the same time, through the pumping action of the coolant generator, the coolant is continuously supplied from one coolant inlet/outlet connector 4 to the cutting head 1 through the elongated body 2 and exits from the other coolant inlet/outlet connector 4 to cool the cutting head. The cooling effect eliminates the need for interruption of operation to clean the cutting head from the tissues stuck thereto. Without this cooling effect, to continue the operation, the surgical apparatus has to be removed from the uterus for cleaning the cutting head and then be inserted back to operation site. However, when the surgical apparatus is inserted back to the operation site, the cutting head may not be put to the exact position where it last acted on. Therefore, the tissues at some positions may be missed and those at other positions may be overtreated and thus, the therapeutic effect is adversely affected.

Having now described several exemplary embodiments of the invention, it should be evident that those skilled in the art may now make numerous uses and modifications of and departures from the specific embodiment described herein without departing from the inventive concept. Therefore, numerous other embodiments of the modifications thereof are contemplated as falling within the scope of the present invention as defined by the appended claims and equivalents thereto.

1. An electrosurgical apparatus for tissue removal, comprising:
a substantially hollow elongated body terminating in a sharp closed cutting head;
an insulative layer covering said elongated body;
said elongated body forming a portion of a coolant path;
a handle located at an end of said elongated body opposite from said cutting head;
coolant inlet/outlet connectors connected to said handle for introducing a coolant into said coolant path of said elongated body and releasing said coolant therefrom; and
a radio-frequency energy input connector connected to said elongated body.

2. The electrosurgical apparatus according to claim 1, wherein said elongated body comprises two metal tubes arranged side-by-side.

3. The electrosurgical apparatus according to claim 2, said cutting head comprises two flat metal tubes arranged side-by-side and sealed at the ends, said metal tubes being in fluid communication with and connected to said two metal tubes of said elongated body.

4. The electrosurgical apparatus according to claim 1, wherein said elongated body comprises two metal tubes with different diameters, one with a smaller diameter being disposed coaxially within the other with a bigger diameter.

5. The electrosurgical apparatus according to claim 1, wherein said insulative layer is a medical insulative paint or an insulative tube surrounding said elongated body.

6. An electrosurgical apparatus for tissue removal, comprising:
a substantially hollow elongated body terminating in a closed cutting head; wherein said elongated body comprises a metal tube which is folded over to form a loop section and a fork section having two legs;
an insulative layer covering said elongated body;
said elongated body forming a portion of a coolant path;
a handle located at an end of said elongated body opposite from said cutting head;
coolant inlet/outlet connectors connected to said handle for introducing a coolant into said coolant path of said elongated body and releasing said coolant therefrom; and
a radio-frequency energy input connector connected to said elongated body.

7. The electrosurgical apparatus according to claim 6, wherein said two legs of said fork section are adhered to each other.

8. The electrosurgical apparatus according to claim 6, wherein said cutting head comprises said loop section of the folded tube.

9. The electrosurgical apparatus according to claim 8, wherein said cutting head is arranged at an angle to said elongated body.

10. The electrosurgical apparatus according to claim 6, wherein said insulative layer is a medical insulative paint or an insulative tube surrounding said elongated body.
